(19) United States
(12) Patent Application Publication
(10) Pub. No.: US 2007/0218566 A1
(43) Pub. Date: Sep. 20, 2007

(54) Method and Device for Improved Purification of a Substance Bound to Paramagnetic Microparticles

(76) Inventors: Roland Barten, Marburg (DE); Marion Kramer, Forchheim (DE); Jurgen Krause, Erlangen (DE); Sandra Strich, Erlangen (DE); Anja Weiland, Poxdorf (DE)

Correspondence Address: FISH & RICHARDSON P.C., PO BOX 1022, MINNEAPOLIS, MN, 55440-1022, US

(21) Appl. No.: 10/580,066

(22) PCT Filed: Nov. 16, 2004
(86) PCT No.: PCT/EP04/12988

§ 371 Date: Feb. 22, 2007

(30) Foreign Application Priority Data
Nov. 20, 2003 (DE) 103 54 351.1

(51) Int. Cl. G01N 33/553

(52) U.S. Cl. 436/526

(57) ABSTRACT

The invention relates to a method for improved cleaning of a first substance which is bound to paramagnetic microparticles. Said microparticles are suspended in a first liquid. The inventive method comprises the following steps: a) microparticles are exposed to a first magnetic field in a first container so that they are securely maintained and are prevented from being washed away by a flow of the first liquid, and b) after step a), at least one part of the first liquid is guided through the line in a first direction of the first line and through another section, and in the second section, said part is exposed to a second or to the first magnetic field in order to hold onto the washed away microparticles. In the section, the surface of the cross section of the first line is enlarged. The second or first magnetic field inside the section has a larger cross-sectional field strength than the first magnetic field inside the first container.

The invention relates to a method for improved purification of a substance bound to paramagnetic microparticles and to a device suitable for carrying out the method.

Magnetic microparticles are used in particular in diagnostic and analytic methods. They have in relation to their mass a large surface area, to which analytes to be detected can be specifically bound by means of a coating. The microparticles can be reversibly immobilized with the aid of a magnetic field. Substances that are not bound can then be separated from the microparticles together with the liquid containing the substances. After that, the microparticles can be washed in a washing liquid and specifically bound substances can be eluted with an elution liquid. A stream of liquid occurring during the separation can have the effect that some of the magnetically immobilized microparticles are suspended and washed away. That leads to a loss of the bound analytes. Microparticles washed away during elution may disturb a subsequent reaction, such as for example a polymerase chain reaction (PCR), because of their iron content. Furthermore, detection methods based on optical properties may be disturbed by the washed-away microparticles, because the microparticles can scatter and absorb light and because they may have a fluorescence. In addition, electrochemical detection methods may be influenced, for example because the iron contained in the microparticles may be reduced as a result. The washed-away microparticles may have the effect of reducing the sensitivity of the diagnostic or analytic method.

In the case of medical applications of the purified substance, washed-away microparticles may have an immunogenic effect or induce thrombus formation. A further problem occurring in medical applications is that the microparticles may clog filters that are used for example for the sterile filtration of solutions to be administered. It is also disadvantageous that the microparticles rebind the purified substance and, as a result, may reduce the available amount of this substance.

U.S. Pat. No. 4,910,148 discloses a device for separating magnetic particles from biological liquids in which the magnetic particles are suspended. The device contains a magnetic plate with a multiplicity of permanent magnets. For separating the magnetized particles, the biological liquid with the magnetized particles suspended therein is moved in a container over the plate. While the magnetized particles are held back by the magnetic plate, the biological fluid is removed by suction.

EP 0 237 549 B1 discloses a separating device for separating magnetic particles from a liquid medium. The separating device contains a separator with a flow chamber with an inlet and an outlet for the liquid medium and also a magnetizing means. Depending on the position of the flow chamber in relation to the magnetizing means, a strong or weak magnetic field forms in the flow chamber. The separating device may comprise a first separator and a second separator of such a construction. As a result, sample throughput can be increased. Furthermore, fractionation of a sample can be achieved by differently adjusted separators.

US 2003/0095897 A1 discloses a method in which a liquid with magnetic particles dispersed therein is passed through a magnetic field, the magnetic particles being arrested. The magnetic field is constant. The release of the magnetically arrested particles takes place by means of a pulsed stream of liquid.

In all the known methods, a disadvantage is that the flow of a liquid which flows past the already magnetically arrested particles can cause these particles to be suspended. As a result, the already magnetically arrested particles can be washed away with the liquid. This entails the aforementioned disadvantages.

The object of the present invention is in particular to eliminate these disadvantages of the prior art. It is intended to provide a method and a device which allow paramagnetic microparticles to be magnetically arrested in a fluidic system, quantitatively as far as possible, and optionally released again. The method and the device are to be simple and inexpensive to implement.

According to the invention, the object is achieved by the features of claims 1 and 13. Expedient embodiments are provided by the features of claims 2 to 12 and 14 to 30.

According to the invention, a method for improved purification of a first substance, which is bound to paramagnetic microparticles, the microparticles being suspended in a first liquid, is provided with the following steps:
a) the microparticles are exposed in a first container to a first magnetic field, to thereby arrest them and prevent them from being washed away with a stream of the first liquid and
b) after step a, at least part of the first liquid is passed in a first direction through a first line, through a portion of the first line, and exposed in the portion to a second magnetic field or once again to the first magnetic field, to thereby arrest microparticles that have nevertheless been washed away, the cross-sectional area of the first line being enlarged in the portion,
the second or first magnetic field within the portion having a greater average field strength than the first magnetic field within the first container.

The substance may comprise cells, molecules or aggregates. The part of the first liquid according to step b is preferably a predominant part of the first liquid or the entire first liquid. When the microparticles are exposed to the first magnetic field in step a, a predominant part of the microparticles suspended in the first liquid is thereby held back in the first magnetic field. The magnetic field may be provided by means of an electromagnet or permanent magnet. The arresting according to step a may take place in part of a container in which the microparticles are suspended in the liquid. The magnet is in this case preferably arranged in such a way that the magnetically arrested microparticles are not deposited at a position in which they can hinder the flow of the liquid through the first line. The first container may also be a chamber intended for magnetically arresting the microparticles, with a supply line and a discharge line for the first liquid.

In the method according to the invention, in step b the flow rate of the stream of the first liquid is reduced by the enlarged cross-sectional area of the first line in the portion. This facilitates the arresting of the microparticles in the portion by the first or second magnetic field. The portion of the line with the enlarged cross-sectional area may be in the form of a chamber. The second or first magnetic field within the portion may have a greater average field strength than the first magnetic field within the first container by the portion having a relatively small volume and this volume altogether being located close to a magnet producing the first or second magnetic field. This allows the average field strength in the portion to be greater than the average field strength in the first container even if the second magnetic field altogether is not stronger than the first magnetic field or the first magnetic field acts both in the container and in the portion. The method allows microparticles that are washed away out of the first container to be arrested almost quantitatively in the portion.

The purification according to the method according to the invention may be performed for sample preparation for carrying out a PCR or method of detection. It may also serve the purpose of purifying the first substance for any other application, for example a medical application. In the applications, the paramagnetic microparticles may represent an undesired contamination.

The microparticles arrested in the portion are preferably returned to the microparticles arrested in step a. For this purpose, after step b, a second and/or further liquid may be passed in a second direction into the portion, and in a direction opposite to the first direction through the first line, an effect of the first and optionally present second magnetic field on the microparticles being discontinued, so that the microparticles arrested in the portion are suspended and at least partially washed back to the microparticles arrested in step a. This allows the loss of paramagnetic microparticles when carrying out washing steps to be significantly reduced and the yield of the first substance that is to be purified to be significantly increased.

The second direction, with which the second and/or further liquid is passed into the portion, may for example be perpendicular to the first direction, so that the second and/or further liquid flows directly onto the held-back microparticles and suspends them. The flowing away of the second and/or further liquid through the first line takes place in a direction opposite to the first direction. The second direction may also be a direction opposite to the first direction. This simplifies the construction of the device, which otherwise would for example have to have a further line for the flowing of the second and/or further liquid into the portion.

The second liquid is generally a washing solution. This may have an identical composition to the first liquid. The second liquid is preferably chosen such that the first substance in it remains bound to the microparticles and further substances or other contaminants are detached from the microparticles or the first substance. The detachment may be achieved for example by the second liquid containing a detergent. The further liquid may be chosen such that the first substance in it is detached from the microparticles. It is generally an elution solution.

The portion is preferably formed in such a way that, when the second and/or further liquid flows in the second direction, turbulences are produced in the portion, so that microparticles deposited there are suspended. This may take place for example by means of a projection arranged correspondingly in the portion, or some other device for guiding a stream of liquid. It is particularly preferred if a second line in the portion has an opening via which the second and/or further liquid is passed into the portion in such a way that turbulences are produced in the portion and microparticles deposited there are suspended.

Steps a and b may be repeated with the second and/or further liquid instead of the first liquid. In this case, the microparticles are largely removed from the second and/or further liquid. If the further liquid is an elution liquid, it may be used after step b directly for a method of detection, such as a PCR. The method of detection is not impaired, or scarcely impaired, by the microparticles, since they are, at least almost, held back quantitatively. Removal of microparticles still contained in the further liquid, for example by means of a filter, is not required. This simplifies the automation of the method, since there would always be the risk of clogging of the filter during filtration, and the clogged filter would then usually have to be manually exchanged.

The first magnetic field may act in a region within the first container. The microparticles may be exposed to the first magnetic field by a permanent magnet being brought up to the region and the portion. A single magnet may be used to produce a magnetic field in which, for example, one pole of the magnet causes a strong magnetic field primarily in the region and the other pole of the magnet causes a strong magnetic field primarily in the portion. The microparticles may also be exposed to the first and second magnetic fields by a permanent magnet being respectively brought up to the region and the portion.

The microparticles preferably have an average diameter of from 50 nm to 50 μm, preferably from 500 nm to 50 μm. Such microparticles have proven to be particularly favorable for the purification of substances. The microparticles may have a coating of glass, silicate, silane, an ion exchanger, a receptor, a ligand, an antigen, an antibody or a nucleic acid.

The invention also relates to a device for carrying out a method according to the invention. The device comprises:
a first container for providing or receiving a first liquid and paramagnetic microparticles,
a first line, opening out into the first container, and
a portion of the first line, which has an enlarged cross-sectional area in comparison with the remaining cross-sectional area of the first line,
a first magnet or a first recess for receiving a first magnet for producing a first magnetic field in a region of the first container and in the portion or
a first magnet or a first recess for receiving a first magnet for producing a first magnetic field in a region of the first container and a second magnet or a second recess for receiving a second magnet for producing a second magnetic field in the portion,
the region, the portion and the first recess or the first magnet and, if present, the second recess or the second magnet being arranged and/or formed in such a way that the first or second magnetic field that is produced or can be produced by the first or second magnet within the portion has a greater average field strength than the first magnetic field that is produced or can be produced by the first magnet within the first container, even if the second magnetic field altogether is not stronger than the first magnetic field.

The region of the first container is an area within the first container and preferably in the vicinity of the mouth of the first line in the first container. The region is preferably arranged in such a way that paramagnetic microparticles magnetically arrested in it do not hinder a liquid flow through the first line.

The magnet may be permanently connected to the device and be formed for example as an electromagnet which can be activated as and when required.

It is particularly preferred for the region, the portion and the first recess or the first magnet to be arranged in such a way that a magnetic field that is produced or can be produced by the first magnet can act or does act both in the region and in the portion. This may be ensured for example by the region and the portion being located on opposite sides of the first recess into which the magnet can be inserted, so that one pole of the magnet can act on the region and the other pole of the magnet can act on the portion.

The magnet is preferably a permanent magnet. To act on the microparticles, it can be brought up to the portion or the region. This is easy to ensure in the case of automated sample preparation. The device itself then does not have to have a magnet.

The portion may be formed as a recess in the first line. The recess may in this case be formed such that it is wide and flat. This allows a high magnetic field strength to be provided in the region of the recess. Furthermore, the flow rate can be slowed as a result. The recess is preferably dimensioned in such a way that the microparticles can be deposited in it without thereby reducing the cross-sectional area of the first line in the region of the recess in comparison with the remaining cross-sectional area of the first line. Such a reduction of the cross-sectional area would bring about a constriction in the first line and, as result, an increase of the flow rate. This could lead to deposited microparticles being washed away.

The portion is preferably formed in such a way that, when a liquid flows in a first direction, a laminar flow can be produced in the portion, and when the liquid flows in a second direction, in particular opposite to the first direction, a turbulent flow can be produced. This allows microparticles that are deposited in the portion to be suspended particularly efficiently and returned to the microparticles deposited in the region.

Preferably, at least one second line branches off from the first line. For example, the first line may for this purpose fork into two lines. The branching or forking may be located in the portion or after the portion in the direction of flow of a liquid flowing from the first container through the first line. An opening of the second line may open out in the portion, the opening being arranged in such a way that liquid flowing through the opening into the portion can cause turbulences in the portion and, as a result, microparticles deposited there can be suspended.

It has proven to be particularly favorable if the first line has a diameter of from 50 μm to 2 mm, with preference from 100 μm to 500 μm. The portion advantageously has a cross-sectional area which is at most three times, preferably at most two times, as large as the cross-sectional area of the first line. This permits an adequate reduction of the flow rate to make depositing of the microparticles possible in the first or second magnetic field. Preferably, the portion has a cross-sectional area of at most 2 mm², preferably 1 mm².

In the device according to the invention, a second container for the provision of a second liquid, a third container for the provision of a further liquid and/or a fourth container for receiving the first and optionally second and/or further liquid may be provided. As a result, it is possible to provide the device as a closed system and to move the first, second and/or further liquid only within the device, without liquid contained in the device being able to penetrate to the outside. This is advantageous in particular whenever an infectious or otherwise at least potentially hazardous material, such as blood for example, is to be processed for purifying the substances. The second line may open out into the second container. Optionally provided further lines may open out into the third or fourth container.

A plunger may be respectively provided in the first, second, third and/or fourth container, which plunger is displaceable therein and by means of which the first, second or further liquid can be moved. This measure also makes it possible to provide the device as a closed system. For moving the liquids in the device, the only external action required is to move the plungers. The first, second, third and/or fourth container may in each case be provided in the form of an exchangeable cartridge, in particular a liquid-filled cartridge. As a result, various first, second or further liquids can be provided for use in the device in a simple way. In addition, the use of cartridges avoids the need for the liquids to be openly handled. This measure is therefore also advantageous with regard to the provision of a device formed as a closed system. Instead of the first, second, third or fourth container, the device may in each case have a recess for receiving the cartridge.

It is preferred for the first, second, third and/or fourth container to be cylindrically formed. This has the advantage, both with regard to the use of cartridges and with regard to the moving of the liquid by means of plungers, that wrongly oriented insertion of the cartridge or the plunger can be largely ruled out on account of the rotational symmetry. The first, second, third or fourth container preferably has a maximum volume of from 50 μl to 50 ml, with preference from 500 μl to 5 ml. If the container contains a plunger, the maximum volume relates to the volume with the plunger withdrawn.

The device is preferably insertable into a unit for sample processing, in particular automated sample processing. The unit and the device are in this case made to match each other. The unit may have, for example, at least one means for displacing the plunger or the plungers in the device. The device may also be formed in such a way that it can be connected to the unit in such a way that liquid can be passed. This allows a sample which contains a substance that is to be purified to be fed to the device from the unit in an automated manner. Furthermore, the purified substance can be further processed in the unit.

The device can be produced particularly inexpensively from a plastic, in particular polycarbonate, preferably by means of an injection-molding process.

The invention is explained in more detail below on the basis of drawings, in which:

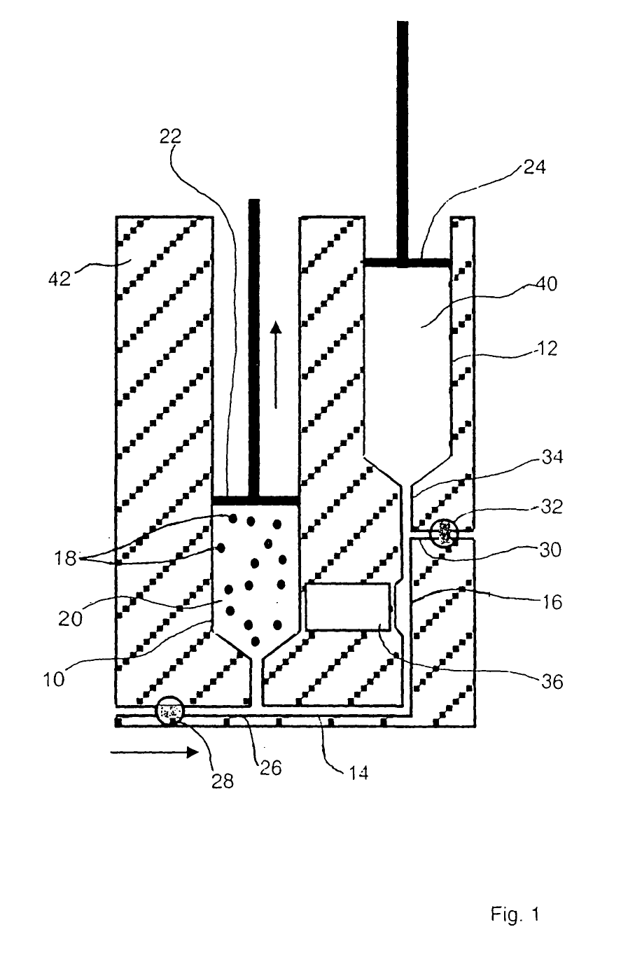

FIG. 1 shows a schematic representation of a device according to the invention in a first working step,

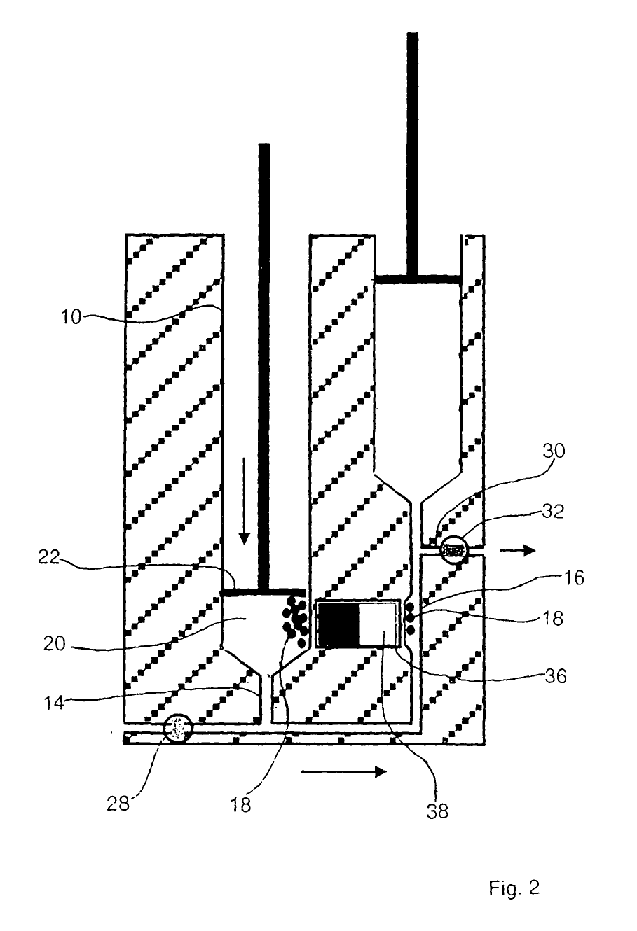

FIG. 2 shows a schematic representation of a device according to the invention at the end of one of the steps a and b of the method according to the invention corresponding to the second working step,

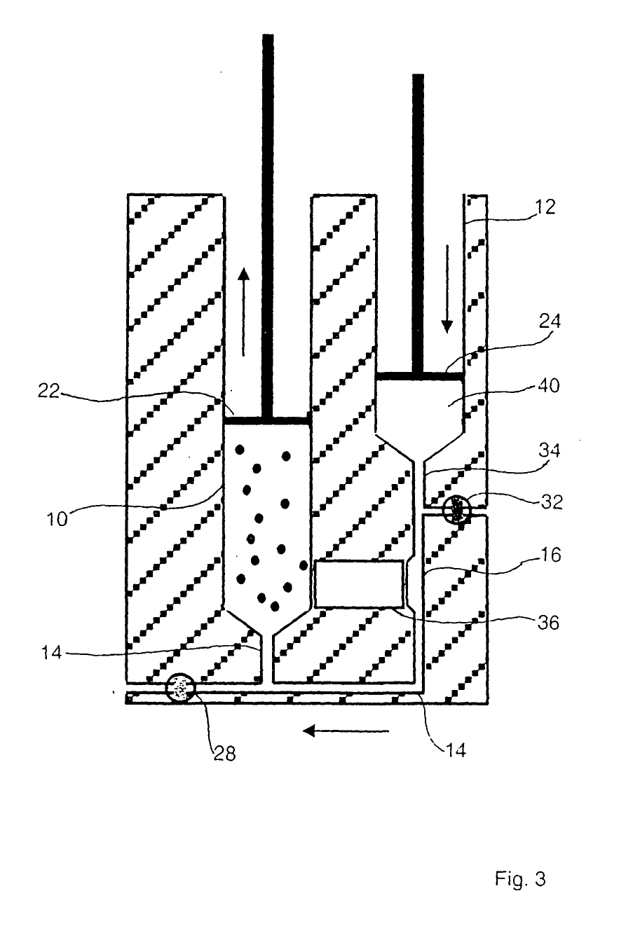

FIG. 3 shows a schematic representation of a device according to the invention at the end of a third working step of the method according to the invention, specified in claim 2,

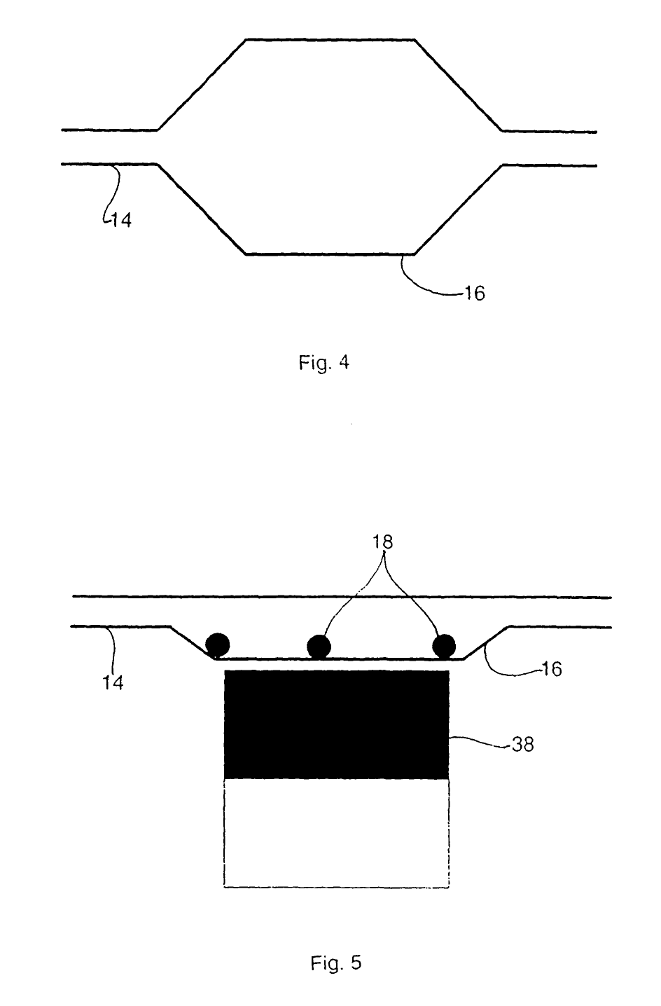

FIG. 4 shows a plan view of a schematic representation of a portion of the first line which has an enlarged cross-sectional area,

FIG. 5 shows a cross section through this portion of the first line with a magnet and paramagnetic microparticles arrested in the portion by the magnetic field of the magnet, and

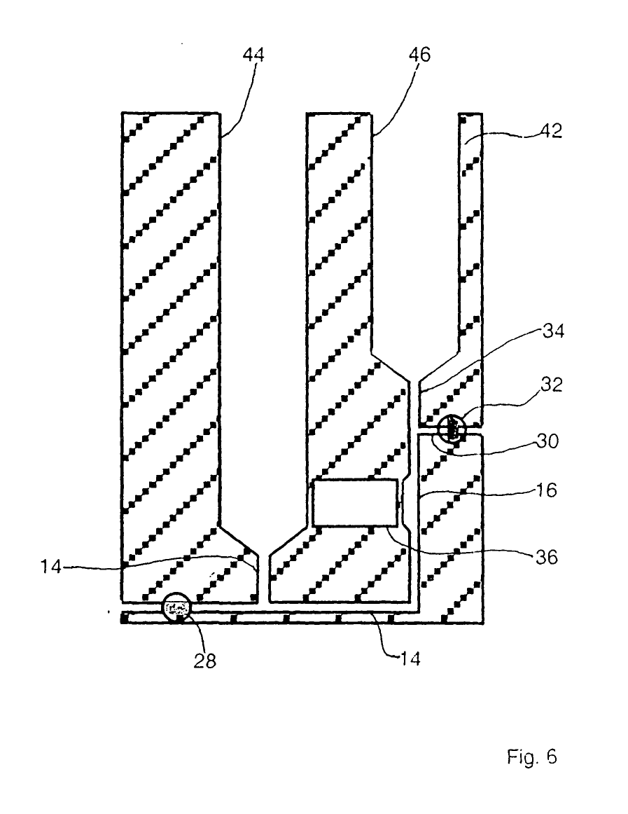

FIG. 6 shows a schematic representation of a further embodiment of the device according to the invention for the insertion of first and second containers in the form of cartridges.

FIG. 1 shows a device according to the invention, formed in a substrate 42, with a first container 10, a second container 12, a first line 14 and a portion 16 with a cross-sectional area that is enlarged in comparison with the first line 14. In the first container 10 there are paramagnetic microparticles 18, which are suspended in a first liquid 20. In the first container 10 and the second container 12 there is in each case a displaceable plunger 22, 24. In a supply line 26 leading to the first line 14 there is a first valve 28, here in the open position. In a further line 30, leading away from the portion 16, there is a second valve 32, here in the closed position. Between the portion 16 and the second container 12 there is a second line 34. Between the portion 16 and the first container 10 there is a first recess 36. For carrying out the method, firstly the first piston 22 is raised. Liquid is thereby sucked out of the supply line 26 into the first container 10 and thereby suspends magnetic microparticles 18 located in it.

In the second working step, represented in FIG. 2, firstly a magnet 38 is inserted into the recess 36. The first valve 28 is closed and the second valve 32 is opened. Subsequently, the plunger 22 is moved downward and, as a result, the first liquid 20 is forced out of the first container 10 through the first line 14, the portion 16 and the further line 30, through the opened second valve 32. Thereby, the magnetic microparticles 18 are arrested in a region of the container and in the portion 16 by a first magnetic field produced by the magnet 38. The first magnetic field has in this case a greater average field strength within the portion 16 than within the first container 10. The reason for this is that the portion 16 has a smaller volume, arranged altogether closer to the magnet 38, than the container 10. As a result, the average distance which a microparticle in the portion 16 may be from the magnet is less than the average distance which a microparticle in the container 10 may be from it.

In a third working step, represented in FIG. 3, the magnet 38 is removed from the recess; 36, so that the paramagnetic microparticles 18 are no longer arrested. The second valve 32 is closed. The second liquid 40, located in the second container 12, is forced through the second line 34, the portion 16 and the first line 14 into the first container 10 by pressing down the plunger 24. At the same time, the plunger 22 is raised. The paramagnetic microparticles 18 are thereby suspended. The paramagnetic microparticles 18 arrested in the portion 16 are thereby washed back into the container 10.

FIG. 4 shows a plan view of the portion 16 of the first line 14, which has an enlarged cross-sectional area in comparison with the first line 14.

FIG. 5 shows a cross section of this portion 16. In the portion 16 there are paramagnetic microparticles 18 arrested by the magnetic field of the magnet 38.

FIG. 6 shows a schematic representation of a further embodiment of the device according to the invention. This comprises a substrate 42, in which a first recess 36 for receiving a magnet and a third recess 44 and a fourth recess 46 for respectively receiving a first and a second cartridge, forming the first container 10 and the second container 12, are provided. The cartridges may in each case contain a plunger. Furthermore, the device has a first valve 28 and a second valve 32, a first line 14, a portion 16, a second line 34 and a further line 30. The substrate 42 may consist of a plastic, in particular plastic processed by means of an injection-molding process.

List of reference numbers

| | |
|---|---|
| 10 | first container |
| 12 | second container |
| 14 | first line |
| 16 | portion |
| 18 | paramagnetic microparticles |
| 20 | first liquid |
| 22, 24 | plungers |
| 26 | supply line |
| 28 | first valve |
| 30 | further line |
| 32 | second valve |
| 34 | second line |
| 36 | first recess |
| 38 | magnet |
| 40 | second liquid |
| 42 | substrate |
| 44 | third recess |
| 46 | fourth recess |

1-30. (canceled)

31. A method for improved purification of a first substance bound to paramagnetic microparticles, wherein the microparticles are suspended in a first liquid, the method comprising the following steps:
a) exposing the microparticles to a first magnetic field in a first container to thereby capture the microparticles and prevent the microparticles from being washed away with the first liquid, wherein the first container is in fluid communication with at least a first line, and
b) passing at least part of the first liquid in a first direction through a portion of the first line and exposing the at least part of the first liquid to a second magnetic field or to the first magnetic field in the portion of the first line such that microparticles not captured in the first container are captured, wherein the cross-sectional area of the portion of the first line is enlarged, wherein the first or second magnetic field in the portion of the first line has a greater average field strength than the first magnetic field in the first container.

32. The method of claim 31, further comprising: discontinuing the first or second magnetic field in the portion of the first line, and passing a second and/or a further liquid through the portion of the first line in a second direction opposite that of the first liquid such that at least some of the microparticles captured in the portion of the line are combined with the microparticles captured in the first container.

33. The method of claim 32, wherein the second liquid is selected such that the first substance remains bound to the microparticles and further substances or other contaminants are detached from the microparticles and/or from the first substance.

34. The method of claim 32, wherein a further liquid is selected such that the first substance is detached from the microparticles.

35. The method of claim 32, wherein the portion of the first line is configured in such a way that when the second and/or further liquid passes in the second direction, turbulence is produced in the portion such that microparticles deposited thereon are disrupted and resuspended in the second and/or further liquid.

36. The method of claim 31, wherein a second line has an opening in the portion of the first line and is configured such that when the second and/or further liquid passes into the portion in the first line, turbulence is produced in the portion of the first line and microparticles deposited thereon are resuspended into the liquid.

37. The method of claim 31, wherein steps a and b are repeated with a second and/or further liquid in place of the first liquid.

38. The method of claim 31, wherein the first magnetic field acts on a region within the first container and the microparticles are exposed to the first magnetic field by a permanent magnet that is brought into contact with the region and the portion of the first line.

39. The method of claim 31, wherein the first magnetic field acts on a region within the first container and the microparticles are exposed to the first and second magnetic fields by permanent magnets that are brought into contact with the region and with the portion of the first line, respectively.

40. The method of claim 31, wherein the microparticles have an average diameter of from 50 nm to 50 μm.

41. The method of claim 31, wherein the microparticles have an average diameter of from 500 nm to 50 μm.

42. The method of claim 31, wherein the microparticles have a coating thereon comprising glass, silicate, silane, an ion exchanger, a receptor, a ligand, an antigen, an antibody and/or a nucleic acid.

43. A device for improving the purification of a first substance bound to paramagnetic microparticles, the microparticles being suspended in a first liquid, said device comprising:
a first container for providing or receiving a first liquid comprising the microparticles,
a first line in fluid communication with the first container via an opening, and
a portion of the first line, wherein the portion of the first line has an enlarged cross-sectional area in comparison with the remaining cross-sectional area of the first line,
a first magnet or a first recess for receiving a first magnet, wherein the first magnet or the first recess is positioned to produce a first magnetic field in a region of the first container and/or in the portion of the first line,
optionally, a second magnet or a second recess for receiving a second magnet, wherein the second magnet or the second recess is positioned to produce a second magnetic field in the portion of the first line,
wherein the container, the portion of the first line and the first recess or the first magnet and, if present, the second recess or the second magnet are arranged in such a way that the magnetic field within the portion of the first line has a greater average field strength than the magnetic field within the first container.

44. The device of claim 43, wherein the first and/or second magnet is a permanent magnet.

45. The device of claim 43, wherein the portion of the first line is a recess in the first line.

46. The device of claim 43, wherein the portion of the first line is formed in such a way that when a liquid flows in a first direction, a laminar flow is produced and when a liquid flows in the opposite direction, a turbulent flow is produced.

47. The device of claim 43, further comprising at least one second line branching off from the first line.

48. The device claim 47, wherein the second line comprises an opening that opens into the portion of the first line, wherein the opening is configured in such a way that liquid flows through the opening into the portion of the first line and causes turbulence in the portion of the first line, thereby disrupting microparticles deposited thereon for the purpose of resuspension.

49. The device of claim 43, wherein the first line has a diameter of from 50 μm to 2 mm.

50. The device of claim 43, wherein the first line has a diameter of from 100 μm to 500 μm.

51. The device of claim 43, wherein the portion of the first line has a cross-sectional area that is, at most, three times larger than the cross-sectional area of the first line.

52. The device of claim 43, wherein the portion of the first line has a cross-sectional area that is, at most, two times larger than the cross-sectional area of the first line.

53. The device of claim 43, wherein the portion of the first line has a cross-sectional area of, at most, 2 mm².

54. The device of claim 43, wherein the portion of the first line has a cross-sectional area of, at most, 1 mm².

55. The device of claim 43, further comprising a second container for the provision of a second liquid and, optionally, a further container for the provision of a further liquid and a fourth container for receiving any of the first, second or further liquid(s).

56. The device of claim 55, wherein the second container comprises a second line, wherein the optional further container comprises a further line, wherein the first, second and/or further lines are in fluid communication with the fourth container.

57. The device of claim 55, wherein the first, second, further and/or fourth container comprises a plunger, wherein the plunger(s) are configured to displace liquid from the respective container(s).

58. The device of claim 55, wherein the first, second, further and/or fourth container(s) is/are configured in the form of a replaceable cartridge.

59. The device of claim 55, wherein the first, second, further and/or fourth container are cylindrical.

60. The device of claim 55, wherein the first, second, further and/or fourth container have a maximum volume of from 50 μl to 50 ml

61. The device of claim 55, wherein the first, second, further and/or fourth container have a maximum volume of from 500 μl to 5 ml.

62. The device of claim 43, wherein the device is configured to be inserted into a sample processing unit.

63. The device of claim 62, wherein said sample processing unit is an automated sample processing unit.

64. The device of claim 63, wherein the unit has at least one means for displacing the one or more plungers of claim 57.

65. The device of claim 43, wherein the device is plastic.

66. The device of claim 65, wherein the plastic is polycarbonate.

67. The device of claim 43, wherein the plastic device is produced by means of an injection-molding process.
